(19) United States
(12) Patent Application Publication
(10) Pub. No.: US 2006/0060198 A1
(43) Pub. Date: Mar. 23, 2006

(54) Method and system of scoring sleep disordered breathing

(75) Inventors: Alonzo C. Aylsworth, Wildwood, MO (US); Lawrence C. Spector, Austin, TX (US)

Correspondence Address: CONLEY ROSE, P.C., P. O. BOX 3267, HOUSTON, TX, 77253-3267, US

(73) Assignee: ACOBA, LLC, Chesterfield, MO (US)

(21) Appl. No.: 11/226,570

(22) Filed: Sep. 14, 2005

Related U.S. Application Data
(60) Provisional application No. 60/610,666, filed on Sep. 17, 2004.
(60) Provisional application No. 60/635,502, filed on Dec. 13, 2004.

(51) Int. Cl. A61M 16/00; A62B 7/00

(52) U.S. Cl. 128/204.23; 128/204.18

(57) ABSTRACT

A method and system of scoring sleep disordered breathing. At least some of the illustrative embodiments are a method comprising sensing an attribute of respiratory airflow of a first breath of a patient, converting the attribute to a volume value proportional to the volume of the air respired by the patient, and determining whether the patient experienced a hypopnea or an apnea by comparing the volume value to a reference value created using a value proportional to the volume of a breath preceding the first breath.

### CROSS-REFERENCE TO RELATED APPLICATIONS

This application claims the benefit of provisional application Ser. No. 60/610,666 filed Sep. 19, 2004 titled “Method and system of sleep data scoring that is insensitive to nasal resistance changes.” This application also claims the benefit of provisional application Ser. No. 60/635,502 filed Dec. 13, 2004 titled “Method and system of producing a scoring bar for diagnosis of sleep disordered breathing.” Each of these applications is incorporated by reference herein as if reproduced in full below.

### STATEMENT REGARDING FEDERALLY SPONSORED RESEARCH OR DEVELOPMENT

Not applicable.

### BACKGROUND OF THE INVENTION

Field of the Invention

A hypopnea may be abnormally slow or shallow breathing. Though the definition varies from country to country, in the United States the generally accepted definition of hypopnea is as defined by the American Academy of Sleep Medicine (AASM) in an article titled, “Sleep-Related Breathing Disorders in Adults: Recommendations for Syndrome Definition and Measurement Techniques in Clinical Research” accepted for publication in April 1999 (hereinafter the Chicago Criteria). The Chicago Criteria defines a hypopnea as a “clear decrease (>50%) from baseline in the amplitude of a valid measure of breathing during sleep. . . . The event lasts longer than 10 seconds . . . .” Baseline comes in two varieties: “the mean amplitude of stable breathing and oxygenation in the two minutes proceeding onset of the event”; or, “the mean amplitude of the three largest breaths in the two minutes preceding the onset of the event.” Thus, a reduction of measured amplitude by greater than 50% (with a corresponding time factor of 10 seconds) comprises a hypopnea event.

An apnea may be a cessation of breathing. The Chicago Criteria does not define apnea events, but being that the Chicago Criteria is the de facto standard for hypopnea, it follows that polysomnographers also use the amplitude method to diagnose apnea events. Though again the definition varies, a reduction of measured amplitude of 80-100% (possibly with a corresponding time factor of, e.g., 10 seconds) may comprise an apnea event. Diagnosis of hypopnea or apnea may be made in the related art by a patient sleeping overnight in a sleep lab.

The Chicago Criteria defines use of a pneumotachometer as the reference standard, but pneumotachometers require a snug-fitting face mask (that covers at least the nose and mouth) that fluidly couples to a flow measurement device. The face mask adversely affects a patient's ability to sleep, and thus less intrusive alternatives are used in sleep labs. In particular, in sleep labs, one or more of the patient's breathing orifices are fluidly coupled to a high precision pressure transducer by way of a single lumen cannula. As the patient inhales the reduced pressure created by the patient's diaphragm to draw in air is sensed by the pressure transducer. Likewise during exhalation increased pressure is sensed by the pressure transducer. The peak (positive and negative) amplitudes of sensed pressure are then used with the Chicago Criteria. Alternatively, a temperature sensing device is placed within the patient's respiratory airflow (e.g., thermocouples which create a voltage based on temperature or a thermal resistors (thermistors) whose resistance changes with temperature). The temperature sensed by the temperature sensing device as the patient exhales in relation to the temperature sensed during inhalation (room temperature) fluctuates. The amplitudes of the temperature swings are then used with the Chicago Criteria.

Using the amplitudes of the pressure sensed and/or amplitudes of the temperature swings, a polysomnographer makes a diagnosis as to the presence of hypopnea and/or apnea events. FIG. 1 shows a plot as a function of time of two illustrative inhalations of a patient. Breath 1 has a particular peak P1, and breath 2 has a particular peak P2. Each of the two waveforms of FIG. 1 could be, for example, the absolute value of the inhalation pressure sensed by a high precision pressure transducer coupled to the patient's nares by way of a single plenum cannula. Because P2 is less that half the value of P1, this illustrative situation would be diagnosed as a hypopnea event in the related art. Relatedly, FIG. 2 shows a plot of inhaled airflow as a function of time for four illustrative total oronasal inhalations, such as may be created using a pneumotachometer. All four breaths illustrated have approximately the same peak amplitude, and thus using the Chicago Criteria no disordered breathing would be diagnosed.

In spite of the attempts to correctly diagnose hypopnea and apnea, many patients are misdiagnosed because of the effects of nasal resistance changes on pressure and temperature sensing devices.

### SUMMARY

The problems noted above are solved in large part by a method and system of scoring sleep disordered breathing. At least some of the illustrative embodiments are a method comprising sensing an attribute of respiratory airflow of a first breath of a patient, converting the attribute to a volume value proportional to the volume of the air respired by the patient, and determining whether the patient experienced a hypopnea or an apnea by comparing the volume value to a reference value created using a value proportional to the volume of a breath preceding the first breath.

Yet still other embodiments are a system comprising a processor, a memory coupled to the processor, a first sensor that senses an attribute of airflow electrically coupled to the processor (the first sensor in operational relationship to a first breathing orifice of a patient), and a second sensor that senses an attribute of airflow electrically coupled to the processor (the second sensor in operational relationship to a second breathing orifice of the patient). The processor calculates a first volume value based on a signal from the first sensor during a first breath (the first volume value proportional to air volume through the first breathing orifice during the first breath), and the processor calculates a second volume value based on a signal from the second sensor during the first breath (the second volume value proportional to air volume through the second breathing orifice during the first breath).

The disclosed devices and methods comprise a combination of features and advantages which enable them to overcome the deficiencies of the prior art devices. The various characteristics described above, as well as other features, will be readily apparent to those skilled in the art upon reading the following detailed description, and by referring to the accompanying drawings.

### BRIEF DESCRIPTION OF THE DRAWINGS

For a detailed description of the preferred embodiments of the invention, reference will now be made to the accompanying drawings in which:

FIG. 1 shows a plot as a function of time of two illustrative inhalations of a patient;

FIG. 2 shows a plot as a function of time of instantaneous inhaled airflow for four illustrative total oronasal inhalations;

FIG. 3 illustrates, in graphical form, the inaccuracies when using a single pressure transducer;

FIG. 4 illustrates, in block diagram form, a device constructed in accordance with embodiments of the invention;

FIG. 5 illustrates a method in accordance with the embodiments of the invention;

FIGS. 6A, 6B, 6C and 6D are a plots as a function of time of the airflow of the four inhalations of FIG. 2, along with scoring bars, in accordance with embodiments of the invention; and

FIGS. 7A, 7B and 7C are plots of as a function of time of responses of an airflow sensor, a pressure sensor, and a temperature sensor for an illustrative respiration, and the characteristics of the various signal proportional to volume.

### Notation And Nomenclature

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct connection. Thus, if a first device couples to a second device, that connection may be through a direct connection, or through an indirect connection via other devices and connections.

Further, use of the terms “pressure,” “applying a pressure,” and the like shall be in reference herein, and in the claims, to gauge pressure rather than absolute pressure.

### DETAILED DESCRIPTION OF THE PREFERRED EMBODIMENTS

The inventors of the present specification have found that using amplitudes of sensed parameters from devices such as high sensitivity pressure transducers and temperature sensing devices (thermocouples, thermal resistors, and piezoelectric devices) leads to misdiagnosis in many patients because of inaccuracy of these devices in sensing air volume, especially when taking into account human physiological affects such as changes in nasal resistance.

FIG. 3 illustrates, in graphical form, the inaccuracies with regard to changes in nasal resistance of using a single pressure transducer fluidly coupled to the breathing orifices of a patient. In particular, FIG. 3 illustrates pressure as a function of time as sensed by a single pressure transducer through a single lumen cannula having three ports, the ports positioned one each proximate to the nostrils and the mouth of the patient. During the period of time represented in the figure, the patient maintained a substantially constant respiration rate and inhaled approximately the same volume with each breath. The period of time 300 is illustrative of sensed pressure for a plurality of breaths of the patient breathing through all three breathing orifices. The period of time 302 is illustrative of sensed pressure for a plurality of breaths of nasal only breathing (with the oral tube nonetheless open to flow). The period of time 304 is illustrative of sensed pressure for a plurality of breaths of nasal only breathing with the oral tube blocked (such as by sealing against the lips, face or tongue, or being pinched closed by position of the head on a pillow), and is also illustrative of the response using a single lumen cannula having only narial ports. Note how the peak amplitudes in period of time 304 increase over periods 300 and 302 in spite of the approximately constant respiration rate and volume. Using the Chicago Criteria, a transition from the blocked oral tube case to a case where the oral tube is open would most likely be scored as at least a hypopnea in spite of approximately constant respiration volume.

Still referring to FIG. 3, the period of time 306 is illustrative of sensed pressure for a plurality of breaths with only one nostril, and with the second nostril port and oral port sealed. A transition from the two blocked tube case to any other case would, under the Chicago Criteria, most likely be scored as at least a hypopnea, and possibly an apnea, in spite of approximately constant respiration rate and volume. The response of the pressure signal in the period of time 306, in view of the other periods of time, deserves closer scrutiny.

Consider a patient coupled to a pressure transducer by way of single lumen (single plenum) nasal cannula, with the patient breathing through both nares. Further consider that with each breath the patient inhales a particular volume of air in a particular time. In a first illustrative case during inhalation, the pressure transducer senses a first pressure indicative of the vacuum developed by the patient's diaphragm to inspire the particular volume in the particular time. Now consider that one naris becomes blocked (e.g., by congestion), representing an increase in the patient's nasal resistance. Because the airflow path to the lungs has decreased in cross-sectional area, the patient's diaphragm develops more vacuum to draw in the particular volume in the particular time. In this second illustrative case, the pressure transducer senses more vacuum during inhalation in spite of the fact that the volume as between the two illustrative inhalations is defined to be the same. The response of the pressure signal in the period of time 306 of FIG. 3 is illustrative of greater amplitude pressure swings caused by changes in nasal resistance.

Now consider two illustrative situations of nasal only breathing with one clogged naris, and then a transition to both nares open to flow, except the sensors used are electrically paralleled temperature sensing devices one each within the nasal airflow. Further consider that with each breath the patient inhales a particular volume of air in a particular time. In the illustrative case of one blocked naris, the airflow through the unblocked naris is faster (for the particular volume and the particular time), and thus the temperature sensing device is exposed to a fast airflow rate. The difference in temperature sensed between inhalation and exhalation in this first case will be a particular value. As the second naris becomes unblocked, the airflow rate is slower (assuming, again, the particular volume and the particular time), and the difference in temperature sensed will be less. Thus, difference in amplitude in sensed temperature as between these two situations will be different in spite of the fact that in these illustrative situations it has been defined that there is no change in the volume of air inhaled by the patient. Chicago Criteria scoring, based on differences in peak amplitude, in these illustrative cases thus may lead to misdiagnosis of a hypopnea and/or an apnea.

The ambient environment also affects temperature sensing devices. Temperature sensing devices move toward a reading of ambient temperature during inhalation, and toward a reading of the temperature of the gas exiting the patient during exhalation. Thus, even if the patient is defined to have constant total oronasal respiratory volume, changes in ambient temperature produce different peak amplitudes, and these changes too could produce misdiagnosis of a hypopnea and/or apnea event.

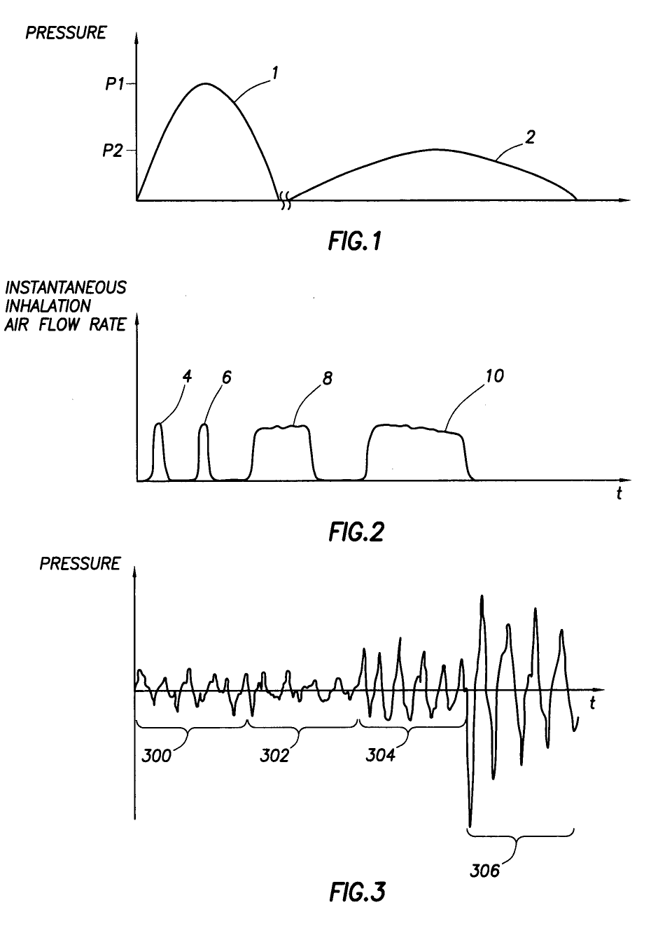

Turning again to FIG. 1 discussed in the Background section, breath 1 has a peak P1 more than twice the peak P2 of breath 2, and with breath 2 following breath 1, breath 2 may be considered indicative of at least a hypopnea event under the Chicago Criteria. However, with the two waveforms depicting pressure as a function of time, the volume of air represented by each of the two waveforms of FIG. 1 (proportional to the area under the two waveforms) is approximately the same. That is, the area under the breath 1 waveform is approximately the same as the area under the breath 2 waveform. Thus, in actuality, no hypopnea event is indicated by the illustrative case of FIG. 1. Turning again to FIG. 2 discussed in the Background section, if the waveforms depict inhaled airflow rate as a function of time, the first breath 4 and the second breath 6 are of significantly lower air volume than the third breath 8 and the fourth breath 10. Thus, the illustrative waveforms could represent a patient experiencing a significant drop in blood-oxygen saturation (proximate in time to breaths 4, 6), and a breakthrough event (breaths 8 and 10), most likely associated with brain arousal and therefore disruption of sleep. Under the Chicago Criteria using peak amplitudes however, no event would be noted.

The various embodiments of the present invention address, at least to some extent, the shortcomings of the related art sleep scoring by determining or calculating at least a portion of the respired air volume, which thus allows scoring based on air volume breath-to-breath to determine whether the patient experienced and apnea or hypopnea. In some embodiments, this may be accomplished by finding the area under the curves of sensed parameters such as pressure or temperature. In alternative embodiments, at least a portion of the airflow of the patient may be sensed, with the air volume calculated for each breath.

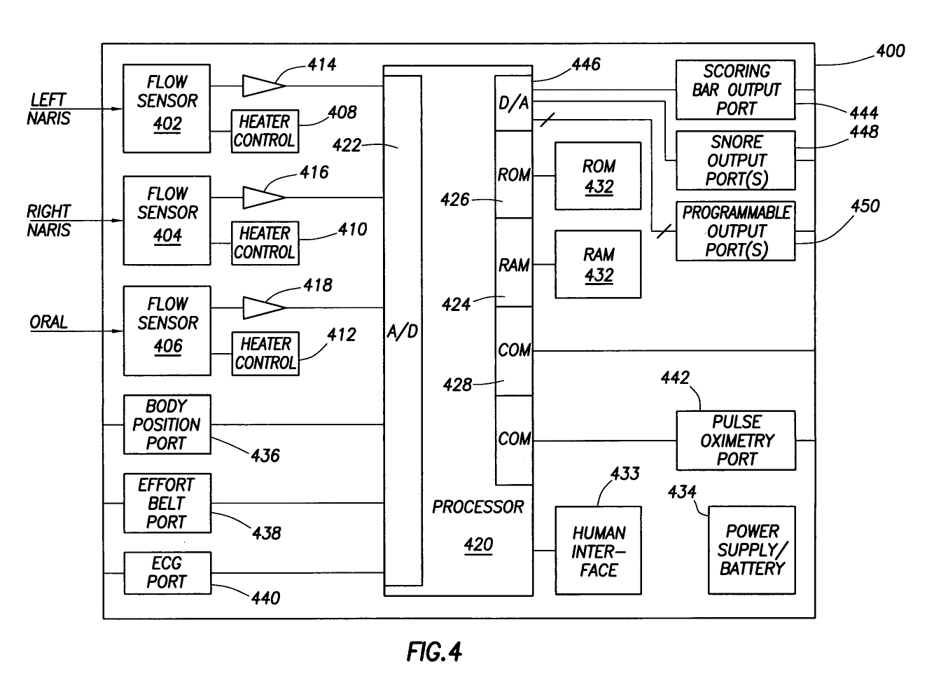

FIG. 4 illustrates, in block diagram form, a sleep study device 400 constructed in accordance with at least some embodiments of the invention. The device 400 may comprise a flow sensor 402 that fluidly couples to a left naris of a patient, possibly by way of a first plenum of a dual lumen cannula (not specifically shown). The device 400 also comprises another flow sensor 404 that couples to a right naris of a patient, possibly by way of a second plenum of the dual lumen cannula. The device may also comprise a third flow sensor 406 which fluidly couples to the mouth of the patient. In accordance with at least some embodiments of the invention, the flow sensors 402, 404 and 406 may be mass flow sensors available from Microswitch (a division of Honeywell Corp.) having part number AWM92100V. However, other mass flow sensors, pressure sensors (such as a Motorola MPXV5004DP pressure transducer) and/or temperature sensing devices (such as thermocouples, thermal resistors and piezoelectric devices) may be used in place of the mass flow sensors. In embodiments using the mass flow sensors noted above, heater control circuits 408, 410 and 412 may be used. Mass flow sensors of differing technology may not require heater control circuits.

The sleep study device 400 of FIG. 4 may also comprise amplifiers 414, 416 and 418 coupled to the flow sensors 402, 404 and 406 respectively. The purpose of amplifiers 414, 416 and 418 is to amplify the output signals propagating from each of the flow sensors. Depending on the type of flow sensors used, amplifiers 414, 416 and 418 may not be needed. In accordance with some embodiments, each flow sensor 402, 404 and 406 produces an output signal that has an attribute that changes proportional to the instantaneous airflow rate. Any attribute of an electrical signal may be used, such as frequency, phase, current flow, or possible a message based system where information may be coded in message packets. In the preferred embodiments each sensor produces an output signal whose voltage is proportional instantaneous airflow rate.

The sleep study device 400 also comprises a processor 420, shown to have an on-board analog-to-digital (A/D) converter 422, on-board random access memory (RAM) 424, on-board read-only memory (ROM) 426, as well as an on-board serial communication port 428. In embodiments where these devices are integral with the processor, the processor may be any of a number of commercially available microcontrollers. Thus, the processor 420 could be a microcontroller produced by Cypress Micro Systems having a part no. CY8C26643. In alternative embodiments of the invention, the functionality of the microcontroller may be implemented using individual components, such as an individual microprocessor, individual RAM, individual ROM, and an individual A/D converter. Random access memory, such as RAM 424, may provide a working area for the processor to temporarily store data, and from which programs may be executed. Read-only memory, such as ROM 426, may store programs, such as an operating system, to be executed on the processor 420. ROM may also store user-supplied programs to read respiratory data and in some situations score the data acquired. Although microcontrollers may have on-board RAM and ROM, some embodiments may have additional RAM 430 and/or additional ROM 432 coupled to the processor 420. The RAM 430 may be the location to which the processor writes sleep data, and in some embodiments where the processor writes an indication of whether a hypopnea and/or apnea was sensed (discussed below). The RAM 430 may be selectively coupled and decoupled from the sleep study device, and sleep data may be transferred to other computers using RAM 430. The RAM 430 may be, for example, a secure digital interface memory card, such as a SDSDB or SDSDJ card produced by SanDisk of Sunnyvale Calif. When using memory such as a secure digital interface memory card, a card reader may be used, such as a card reader part number 547940978 manufactured by Molex Incorporated. Alternatively, the sleep data may be transferred to external devices by way of digital communications, such as through the communications port 428.

The sleep study device may also comprise a human interface 433 coupled to the processor 420. The human interface may comprise a data entry device, such as a full or partial keyboard, along with a display device, such as liquid crystal display. The sleep study device 400 may also comprise a power supply 434. In accordance with at least some embodiments of the invention, the power supply 434 may be capable of taking alternating current (AC) power available at a standard wall outlet and converting it to one or more direct current (DC) voltages for use by the various electronics within the system. In alternative embodiments the sleep study device 400 may be portable, and thus the power supply 434 may have the capability of switching between converting the AC wall power to DC, drawing current from on-board or external batteries, and converting to voltages needed by the devices within the sleep study device. In yet further embodiments, the power supply 434 may be housed external to the sleep study device 400.

Still referring to FIG. 4, a sleep study device 400 in accordance with embodiments of the invention may also couple to various other devices to aid in performing diagnosis of hypopnea and/or apnea events. For example, in some embodiments the sleep study device 400 may have a body position port 436 coupled to the processor 420 by way of the A/D converter 422. The body position port 436 may couple to any commercially available body position indicator, such as a body position indicator having part no. 1664 produced by Pro-Tech Services, Inc. of Mukilteo, Wash. The processor, executing a program, may write body position data to the RAM 424 and/or RAM 430 for later analysis, or may use the body position indication in determining whether the patient's hypopnea and/or apena events are body position dependant.

Some embodiments may also comprise an effort belt port 438 electrically coupled to the processor 420 by way of the A/D converter 422. An effort belt, strapped around a patient's chest, measures increases and decreases in chest circumference as an indication of the patient's breathing effort. Thus, the effort belt port 438 may couple to any commercially available effort belt, such as an effort belt having part no. 1582 produced by Pro-Tech Services, Inc. of Mukilteo, Wash. In addition to (or in place of) the effort belt around the patient's chest, an effort belt may also be strapped around the patents abdomen. In case where two efforts belts are used, an additional effort belt port (not specifically shown) would be used. The processor, executing a program, may write effort data to the RAM 424 and/or RAM 430 for later analysis, or may use the effort indication in determining and/or confirming whether the patient experienced hypopnea and/or apnea events.

Some embodiments may also comprise an electrocardiograph (ECG) port 440 electrically coupled to the processor 420 by way of the A/D converter 422. An ECG analysis provides information on electrical potentials that occur during the patient's heart beat. Thus, the ECG port 440 may couple to any commercially available ECG device. The processor, executing a program, may write ECG data to the RAM 424 and/or RAM 430 for later analysis, or may use the ECG data in determining and/or confirming whether the patient experienced hypopnea and/or apnea events.

Some embodiments may also comprise a pulse oximetry port 442 electrically coupled to the processor 420 by way of the communication port. While FIG. 4 shows the pulse oximetry port 442 coupled to a separate communication port, communication port 428 may serve a dual function, communication with other computers and facilitating communication to an attached pulse oximetry device. A pulse oximeter provides information as to the patient's heart rate and blood oxygen saturation. Thus, the pulse oximetry port 442 may couple to any commercially available pulse oximeter device, such as a Nonin OEMIII pulse oximeter part no. 4518-000. The processor, executing a program, may write pulse and blood oxygen saturation data to the RAM 430 and/or RAM 424 for later analysis, or may use the pulse and blood oxygen saturation data in determining and/or confirming whether the patient experienced hypopnea and/or apnea events. Thus operating as a stand-alone unit, the sleep study device 400 may observe a patient's respiration, and make a diagnosis as the presence of absence of hypopneas and/or apneas. Having described the sleep study device 400, attention now turns to a method of using the device in accordance with embodiments of the invention.

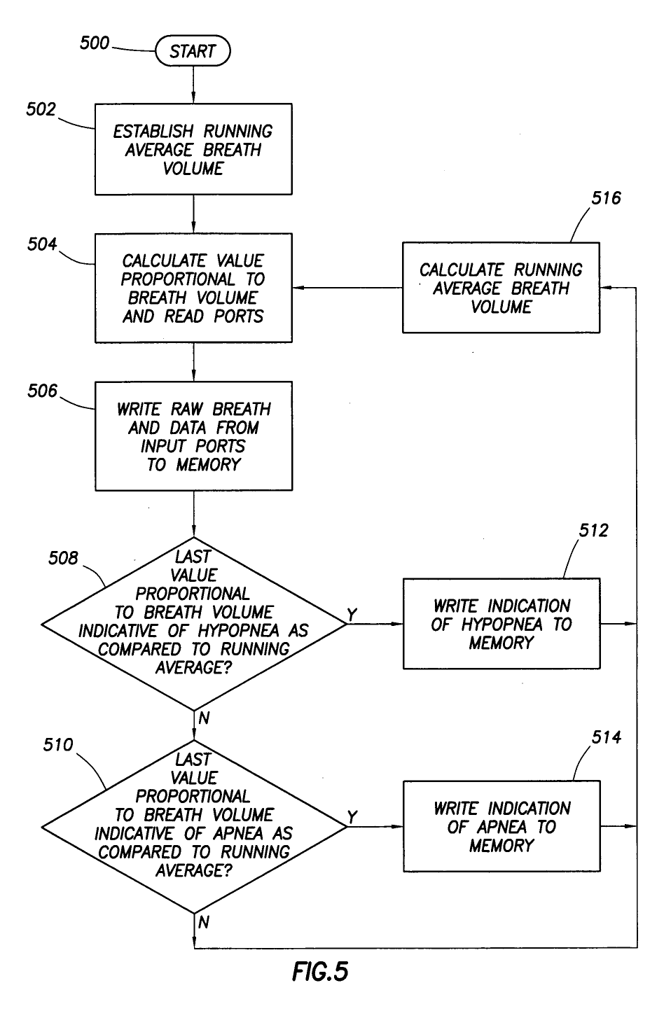

FIG. 5 illustrates a flow diagram of a method that may be implemented by the sleep study device 400. In particular, the process may start (block 500), possibly by a patient or sleep study attendant arming the sleep study device 400. The next step in the illustrative process may be establishing a running average breath volume (block 502), possibly by averaging breath volume (either inhalation volume, exhalation volume, or both) for predetermined period of time when the patient is not experiencing breathing abnormalities. In some embodiments, the predetermined period of time may be two minutes, just as the patient is falling asleep. Other time periods for the predetermined period, and other times for obtaining the initial average, may be equivalently used.

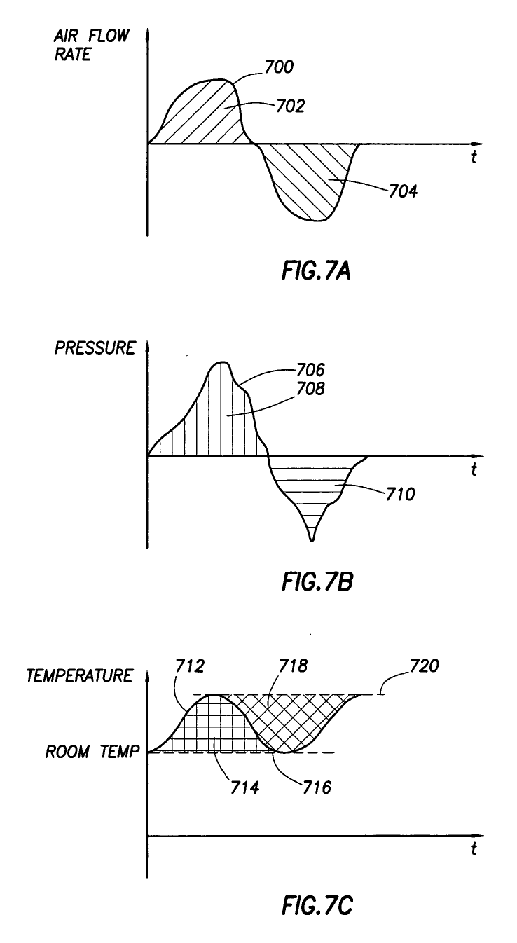

Next, the processor 420 calculates a value proportional to breath volume (e.g., inhalation volume, exhalation volume, or combined volume), and reads data from the various input ports (block 504). Calculating the value proportional to breath volume may involve calculating a value for each breathing orifice, and then summing the values of each breathing orifice. In embodiments using mass flow sensors, calculating the value proportional to volume may involve determining an area between a sensed airflow signal and an axis at zero flow. For example, FIG. 7A shows an illustrative airflow signal 700 as a function of time. Calculating a value proportional to breath volume may thus involve determining the area 702 between the inhalation portion of the airflow signal 700 and the zero flow axis, the determining such as by integration of the airflow signal 700 with respect to time. Alternatively, the area 704 between the exhalation portion of the airflow signal 700 and the zero flow axis may be determined.

In embodiments measuring pressure (vacuum) created by the patient's diaphragm proximate to each breathing orifice, calculating a value proportional to breath volume may involve determining an area between the pressure output signal and an axis at zero gauge pressure. For example, FIG. 7B shows an illustrative pressure signal 706 as a function of time. Calculating a value proportional to breath volume may thus involve determining the area 708 between the inhalation portion of the pressure signal 706 and the zero gauge pressure axis, such as by integration of the pressure signal 706 with respect to time. Alternatively, the area 710 between the exhalation portion of the pressure signal 700 and the zero gauge pressure axis may be determined.

In the case of temperature sensing devices such as thermocouples, thermal resistors and piezoelectric devices, calculating a value proportional to breath volume may involve determining an area between the temperature output signal and an axis being the peak (high or low) temperature sensed. For example, FIG. 7C shows an illustrative temperature signal 712 as a function of time. Calculating a value proportional to breath volume may thus involve determining the area 714 between the exhalation portion of the temperature signal 712 and an axis 716 being the lowest temperature (room temperature), such as by integration of the temperature signal 712 with respect to time and taking into account the offset. Alternatively, the area 718 between the inhalation portion of the pressure signal 712 and an axis 720 being the highest temperature sensed may be determined.

In yet still further embodiments, calculating a value proportional to breath volume may be accomplished using the signal read at the effort belt port 438. As discussed above, effort belts produce a signal proportional to the circumference spanned by the belt. Breathing by a patient produces a somewhat sinusoidal waveform similar to that of FIG. 7C, except that a complete respiration would be illustrated by half the sine wave with the end of an inhalation at a maxima of the circumference length waveform, and the end of an exhalation at the minima of the circumference length waveform. The value proportional to inhalation volume in these cases may thus be calculated as the area between circumference length waveform and an axis being the smallest circumference, calculated in time from the minima (inhalation start) and the maxima (inhalation end). Likewise, the value proportional to exhalation volume would be calculated as the area between the circumference length waveform and an axis being the smallest circumference, calculated in time from the maxima to the minima.

Regardless of the precise method in which a value proportional to breath volume is determined, the next step may be writing the raw breath data and the various values from the input ports (e.g., input ports 436, 438, 440 and 442) to memory (block 506), such as the removable memory 430 (of FIG. 2). Writing the raw data may allow later independent confirmation of the hypopnea/apnea analysis, and thus is not strictly required. Thereafter, a determination is made as to whether the current value proportional to breath volume as compared to the running average is indicative of a hypopnea (block 508). In some embodiments, a hypopnea may be indicated when there is a reduction in breath volume by approximately 50-80% over the running average breath volume (established initial at block 502, and as we shall see also at block 516). Some definitions of hypopnea, e.g., that of Medicare, may also require that the reduced breath volume be present for approximately 10 seconds and further be accompanied by a reduction in blood oxygen saturation by approximately 4% or more. Thus, the determination at block 508 may also be accompanied by a reading of the patient's blood oxygen saturation, possible through the pulse oximetry port 442 (FIG. 4). If the sleep study device 400 detects a hypopnea, an indication of the hypnonea is written to the memory (block 512).

If no hypopnea is detected, the next step is a determination of whether the current value proportional to breath volume as compared to the running average is indicative of an apnea (block 510). In some embodiments, an apnea may be indicated when there is a reduction in breath volume by approximately 80-100% in relation to the running average breath volume. Some definitions of apnea, e.g., that of Medicare, may also require that the reduced breath volume be present for approximately 10 seconds and further be accompanied by a reduction in blood oxygen saturation by approximately 3% or more. Thus, the determination at block 510 may also be accompanied by a reading of the patient's blood oxygen saturation, possible through the pulse oximetry port 442 (FIG. 4). If the sleep study device 400 detects an apnea, an indication of the apnea is written to the memory (block 514). Regardless of the whether a hypopnea or apnea event is detected, or no breathing abnormalities are detected, the next step is calculating a new running average breath volume using the calculated volume of the last breath (block 516). In accordance with at least some embodiments, the running average breath volume uses breath volume data from the last two minutes; however, longer or shorter periods may be used to calculate the running average breath volume. Moreover, in some embodiments breaths with established hypopnea and/or apnea events may be excluded from the running average calculation.

Referring again to FIG. 4, the various embodiments described to this point could operate as a standalone unit, possibly being portable and used in a patient's home. Other uses for the sleep study device may be in a dedicated sleep lab, with the sleep study device gathering data and providing the data (in various forms) to other equipment. For example, the sleep study device 400 may couple to and communicate using packet-based messages with other equipment by way of the communications port 428. The sleep study device 400 may send some or all the raw data, various values from the input ports (e.g., ports 436, 438, 440 and 442), indications of detected hypopnea and/or apnea events, and/or the scoring bar data (discussed below) by way of the communications port 428. In addition to, or in place of, the communications through communications port 428, the sleep study device may drive selected analog data through various output signal ports coupled to the digital-to-analog (D/A) converter 446. For example, the processor 420 may calculate and drive output signals to the programmable output ports 450 (only one shown) with one of: left naris instantaneous airflow rate; right naris instantaneous airflow rate; the combined left naris and right instantaneous airflow rate; the difference between the instantaneous left and right naris airflow rate; the instantaneous oral airflow rate; combined instantaneous oral, left naris and right naris airflow rate; instantaneous oral airflow rate minus the combined left and right naris instantaneous airflow rate; combined instantaneous oral and left naris airflow rate; combined instantaneous oral and right naris airflow rate; instantaneous oral airflow rate minus the left naris instantaneous airflow rate; instantaneous oral airflow rate minus the right naris instantaneous airflow rate; snore signal of the left naris; snore signal of the right naris; snore signal detected at the mouth; or combined left and/or right and/or oral snore signals. Any of these signals may be useful to a polysomnographer in performing manual scoring of sleep data, or verifying automatic scoring.

In situations where the sleep study device 400 is used in conjunction with other equipment and/or in a dedicated sleep lab, the device 400 may also generate what will be termed “scoring bars” which a polysomnographer and/or a computer can use to perform sleep scoring in accordance with the amplitude-based Chicaco Criteria. In particular, for each respiration the processor 420 calculates a value proportional to breath volume, and produces a scoring bar output signal which could be delivered to other equipment by way of communications port 428, but preferably is driven to scoring bar output port 444 by way of D/A converter 446. In some embodiments the processor produces the scoring bar output signal whose amplitude is proportional to the breath volume, and with a constant time width. Alternatively, the scoring bar amplitude could be constant, with the time width proportional to breath volume, but such an output signal could not be easily scored under the amplitude-based Chicago Criteria. Further still, the scoring bar output signal could have a time width proportional to some other parameter, such as blood-oxygen saturation or breath rate.

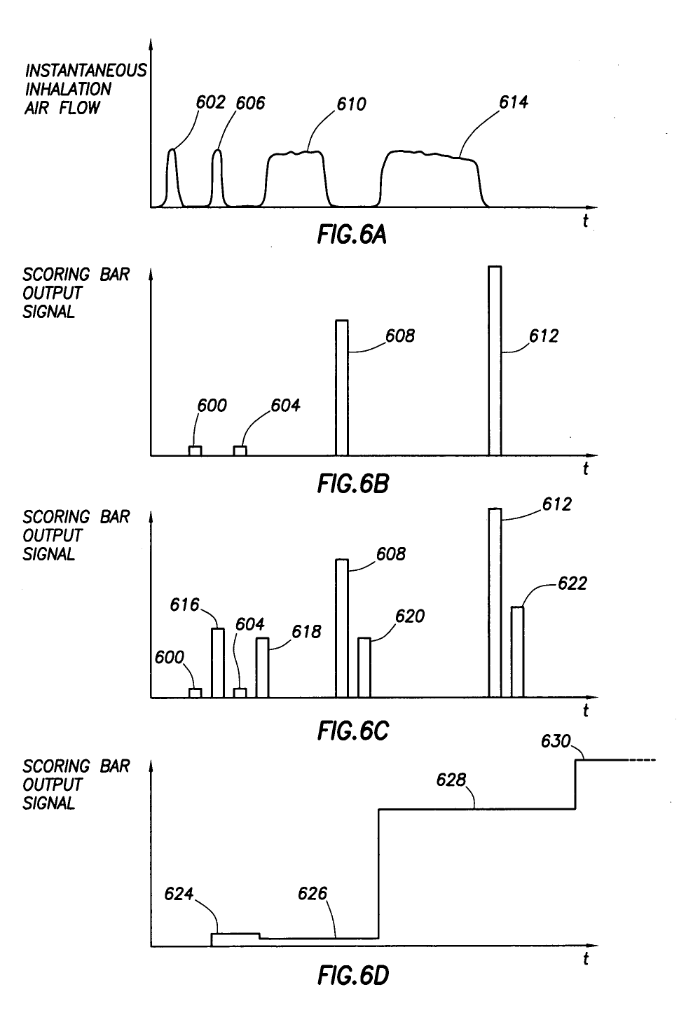

FIG. 6A shows airflow rate as a function of time of the four inhalations of FIG. 2, except in this case the waveforms would be produced by summing the individual flow sensor signals. FIG. 6B, plotted on a corresponding time-axis but on a different y-axis than FIG. 6A, shows four scoring bars in accordance with embodiments of the invention. In each case the scoring bar follows, just slightly in time, the completion of an inhalation, and the delay in producing the scoring bars is attributable to the time it takes processor 420 (of FIG. 4) to compute parameters indicated. In particular, the amplitude of illustrative scoring bar 600 is proportional to the volume represented by waveform 602. Likewise, the amplitude of scoring bar 604 is proportional to the volume represented by waveform 606. The amplitude of scoring bar 608 is proportional to the volume represented by waveform 610. Likewise the amplitude of scoring bar 612 is proportional to the volume represented by waveform 614. Thus it is seen that the scoring bars 600, 604, 608 and 612 can be scored using the amplitude-based Chicago Criteria, either by a polysomnographer or a computer, and that such scoring would be significantly more accurate than an amplitude-based Chicago Criteria scoring of the waveforms 602, 606, 610, and 614 alone.

Some embodiments of the invention, in addition to the scoring bars, also produce other waveforms on the same output signal port 444 as the scoring bars. In particular, in some embodiments the processor 420 also generates a reference or running average bar, which is proportional to a running average calculated breath volume, and which running average bar is driven before or after driving the scoring bar to the output signal port 444. FIG. 6C, plotted on a corresponding time-axis but on a different y-axis than FIGS. 6A and 6B, shows a plot as a function of time of the scoring bars and running average bars in accordance with these alternative embodiments. In particular, running average bar 616 may represent the mean respiratory air volume over the last two minutes. Thus, a polysomnographer and/or a computer need only compare the scoring bar 600 to the running average bar 616 to score the presence of hypopnea or apnea. Likewise, a polysomnographer and/or computer need only compare the scoring bars 604, 608 and 612 to the running average bars 618, 620 and 622 respectively to score the presence of hypopnea or apnea.

Still referring to FIG. 6C, the running average bar 618 may represent the mean respiratory air volume over the last two minutes (including in this illustrative case the volume represented by scoring bar 600, thus accounting for the drop in amplitude from scoring bar 618). As discussed with respect to FIG. 5, in some embodiments reduced inhalations associated with hypopneas or apneas may not be included in the mean or running average respiratory air volume calculation. Thus, running average bar 620 may represent a running average over the last two minutes, but not including the air volume associated with scoring bars 600 and 604 (as these scoring bars may be indicative of an event), accounting for why there is no drop in amplitude as between running average bars 618 and 620. However, running average bar 622 illustrates an increase in the running average attributable to scoring bar 608.

The illustrative running average bars of FIG. 6C are shown to have the same, and in this case arbitrary, time or x-axis width. In alternative embodiments, the time width of the running average bars may be greater or shorter than those of the scoring bars, possibly to help discern the two. In yet other embodiments, the time width of the running average bars may also be a function of other parameters of interest, such as running average breath rate, running average blood-oxygen saturation, or possibly a time-width indication of the snore component of a patient's breathing.

In yet still further alternative embodiments, the scoring bars produced by the processor 420 for a particular inhalation may be driven and span the entire period of the next respiration (inhalation and exhalation). FIG. 6D, plotted on a corresponding time-axis but on a different y-axis than FIGS. 6A, 6B and 6C, shows a plot as a function of time of the scoring bars in accordance with these alternative embodiments. In particular, section 624 has a height the same as scoring bar 600 (proportional to the volume of inhalation 602), but in this case the width spans the period of the next respiration (which comprises the inhalation 606). Likewise, section 626 has a height the same as scoring bar 604, but in this case the width spans the period of the next respiration (which comprises inhalation 610). Sections 628 and 630 are similarly related to scoring bars 608 and 612 respectively. In yet still further alternative embodiments concerned primarily with inhalation volume, the scoring bars may span the period of time starting just after the current inhalation (with the scoring bar driving to its next value as soon as that value is calculated) and holding until the end of the next inhalation. The various embodiments are not limited, however, just to producing scoring bars and/or running average bars, as other manifestations of sleep disordered breathing may be of interest, particularly snoring.

The period of a breath, possibly measured beginning when the patient starts an inhalation and ending just as the patient completes exhalation, may be several seconds long, and in some cases of breathing during relaxation or deep sleep may be ten seconds or more. Breathing frequency, being the inverse of the breathing period, may thus be as slow as 0.1 cycles per second (Hertz). Snoring, on the other hand, may be a relatively rapid air volume undulation that occurs simultaneously with inhalation, possibly having a frequency in the 15-30 Hertz range. A device 400 in accordance with embodiments of the invention may also produce a snore output signal 448 by band-pass or high-pass filtering some or all of the signals created by the flow sensors 402, 404 and 406. The snore output signal 255 port may couple to a data acquisition system within a sleep lab.

The above discussion is meant to be illustrative of the principles and various embodiments of the present invention. Numerous variations and modifications will become apparent to those skilled in the art once the above disclosure is fully appreciated. For example, using the device 400 with a nasal cannula only a portion of the total respiratory volume will be detected; however, the various techniques described to diagnose hypopnea and apnea work equally well even when only a portion of the total volume is detected. In alternative embodiments, a nasal mask, or a system comprising nasal pillows to seal to the nostrils, may be used such that substantially all the respiratory volume is measured, and this too falls within the contemplation of the invention. Thus, in this description and in the claims the terms “volume” and “total volume” may mean measured volume, whether that measured volume comprises some or all the respired volume. In the various embodiments described above, the signal processing to create the signals to drive to the illustrative snore output port 448 and programmable output ports 450 is shown to be done by way of processor 420 and/or a dedicated digital signal processor; however, this processing may alternatively be done with discrete components without departing from the scope and spirit of the invention. Further still, while the scoring bar signal (and possibly running average bar signal) are described as being driving to particular port, in some embodiments the sleep study device may drive those signals directly to an attached display, such as a display associated with the human interface 433. It is intended that the following claims be interpreted to embrace all such variations and modifications.

What is claimed is:

1. A method comprising:
sensing an attribute of respiratory airflow of a first breath of a patient;
converting the attribute to a volume value proportional to the volume of the air respired by the patient; and
determining whether the patient experienced a hypopnea or an apnea by comparing the volume value to a reference value created using a value proportional to the volume of a breath preceding the first breath.

2. The method as defined in claim 1 further comprising:
wherein sensing further comprises sensing airflow rate using a mass flow sensor fluidly coupled within the airflow of a breathing orifice of the patient to create a sensed airflow signal; and
wherein converting further comprises calculating the volume value from the sensed airflow signal.

3. The method as defined 2 wherein calculating further comprises determining the area between the sensed airflow signal and an axis at zero flow.

4. The method as defined 2 wherein calculating further comprises determining the area between the sensed airflow signal during inhalation and the axis at zero flow.

5. The method as defined in claim 1 further comprising:
wherein sensing further comprises sensing pressure using a pressure transducer fluidly coupled to a breathing orifice of the patient to create a pressure output signal; and
wherein converting further comprises calculating the volume value from the pressure output signal.

6. The method as defined in claim 5 wherein calculating further comprises determining an area between the pressure output signal and an axis at zero gauge pressure.

7. The method as defined in claim 5 wherein calculating further comprises determining an area between the pressure output signal during inhalation and the axis at zero gauge pressure.

8. The method as defined in claim 1 further comprising:
wherein sensing further comprises sensing temperature using a temperature sensing device fluidly coupled within the airflow of a breathing orifice of the patient to create a temperature output signal; and
wherein converting further comprises calculating the volume value from the pressure output signal.

9. The method as defined in claim 8 wherein calculating further comprises determining an area between the temperature output signal and an axis at a peak measured temperature.

10. The method as defined in claim 8 wherein calculating further comprises determining an area between the temperature output signal during inhalation and the axis being a highest exhalation temperature.

11. The method as defined in claim 8 further comprising sensing using one device selected from the group: a thermocouple; a thermal resistor; and a piezoelectric device.

12. The method as defined in claim 1 wherein determining further comprises:
producing a reference bar waveform having an amplitude and a time width, wherein the amplitude is proportional to the reference value;
producing a scoring bar waveform having an amplitude and a time width, wherein the amplitude of the scoring bar is proportional to the volume value; and
ascertaining a difference in amplitude between the scoring bar waveform and the reference bar waveform.

13. The method as defined in claim 12 wherein producing the scoring bar further comprises producing the scoring bar wherein time width is one selected from the group: a predetermined constant, the period of a breath of the patient, the patient's blood-oxygen saturation, and the patient's breath rate.

14. The method as defined in claim 12 wherein producing the reference bar waveform further comprising producing the reference bar waveform with the amplitude proportional to an average volume of a plurality of breaths preceding the first breath.

15. The method as defined in claim 12 wherein producing the reference bar waveform further comprises producing the reference bar waveform wherein the time width is one selected from the group: a predetermined constant, frequency of a snore component of the patient's breathing, and amplitude of the snore component of the patient's breathing.

16. A system comprising:
a processor;
a memory coupled to the processor; and
a first sensor that senses an attribute of airflow electrically coupled to the processor, the first sensor in operational relationship to a first breathing orifice of a patient;
a second sensor that senses an attribute of airflow electrically coupled to the processor, the second sensor in operational relationship to a second breathing orifice of the patient;
wherein the processor calculates a first volume value based on a signal from the first sensor during a first breath, the first volume value proportional to air volume through the first breathing orifice during the first breath; and
wherein the processor calculates a second volume value based on a signal from the second sensor during the first breath, the second volume value proportional to air volume through the second breathing orifice during the first breath.

17. The system as defined in claim 16 wherein the processor calculates a breath volume value based on the first and second volume values.

18. The system as defined in claim 17 wherein the processor determines whether the patient experienced a hypopnea or an apnea by comparison of the breath volume to a previous breath volume calculated using a value proportional to air volume of a previous breath.

19. The system as defined in claim 18 further comprising:
a blood oxygen input signal electrically coupled to the processor, the blood oxygen input signal couples to a blood oxygen sensor that senses blood oxygen saturation of the patient;
wherein the processor uses a blood oxygen saturation value to determine whether the patient experienced a hypopnea or an apnea during the plurality of breaths.

20. The system as defined in claim 17 further comprising:
wherein the memory is selectively detachable from the system; and
wherein the processor writes an indication to the memory if a hypopnea or apnea was sensed.

21. The system as defined in claim 17 wherein the processor generates a scoring bar signal having an amplitude proportional to the breath volume value.

22. The system as define din claim 21 wherein the processor generates the scoring bar signal having a time width being one selected from the group: a predetermined constant, the period of a breath of the patient, the patient's blood-oxygen saturation, and the patient's breath rate.

23. The system as defined in claim 21 wherein the processor also generates a reference bar signal having an amplitude proportional to air volume of a previous breath.

24. The system as defined in claim 23 wherein the processor generates the reference bar signal having a time width being one selected from the group: a predetermined constant, frequency of a snore component of the patient's breathing, and amplitude of the snore component of the patient's breathing.

25. The system as defined in claim 21 further comprising:
an output signal port coupled to the processor;
wherein the processor drives the scoring bar signal to the output signal port.

26. The system as defined in claim 25 wherein the output signal port is an analog output signal port.

27. The system as defined in claim 21 further comprising:
a display device coupled to the processor; and
wherein the processor drives the scoring bar signal to the display device.

28. The system as defined in claim 21 further comprising:
wherein the memory is selectively detachable from the system; and
wherein the processor provides the breath volume to other devices by writing the first and second volume values to the detachable memory.

29. The system as defined in claim 17 further comprising:
wherein the first sensor is a first air mass flow sensor that fluidly couples to the first naris of the patient;
wherein the second sensor is a second air mass flow sensor that fluidly couples to a second naris of the patient;
wherein the processor determines a breath volume based on signals from both the first and second air mass flow sensor.

30. The system as defined in claim 29 further comprising:
a third air mass flow sensor electrically coupled to the processor, the third air mass flow sensor fluidly couples to the patient's mouth;
wherein the processor determines the breath volume based on signals from the first, second and third air mass flow sensors.

31. The system as defined in claim 17 further comprising:
wherein the memory is selectively detachable from the system; and
wherein the processor provides the first and second volume values to other devices by writing the first and second volume values to the detachable memory.
